(19) United States
(12) Patent Application Publication
(10) Pub. No.: US 2005/0079134 A1
(43) Pub. Date: Apr. 14, 2005

(54) Contact migration behavior predicting method

(75) Inventors: Yoshihide Matoba, Osaka (JP); Ayumu Inoue, Nishinomiya-shi (JP)

Correspondence Address: FITCH, EVEN, TABIN & FLANNERY, P. O. BOX 65973, WASHINGTON, DC, 20035, US

(73) Assignee: Sumitomo Chemical Company, Limited, Tokyo (JP)

(21) Appl. No.: 10/960,507

(22) Filed: Oct. 8, 2004

(30) Foreign Application Priority Data
Oct. 9, 2003 (JP) P2003-350446

(51) Int. Cl. A61K 49/00; G06F 19/00; G01N 33/48; G01N 33/50

(52) U.S. Cl. 424/9.1; 702/19; 702/22

(57) ABSTRACT

The invention provides a method for estimating the dermal transfer efficiency of a chemical substance comprising a step of determining a calculated value for the dermal transfer efficiency for the time elapsed after use of the chemical substance, based on a formula wherein the temporal change in the dermal transfer efficiency is expressed as a function dependent on the −0.5 power of the time, or on a computer program incorporating the formula, and a step of establishing the calculated value determined in the previous step as the estimated dermal transfer efficiency.

### BACKGROUND OF THE INVENTION

1. Field of the Invention

The present invention relates to a method for predicting the contact migration behavior of chemical substances, and more particularly, it relates to a method of estimating the dermal transferable residue of a chemical substance such as a pesticidal compound and a method of estimating the dermal transfer residue of the chemical substance, based on temporal change in the dermal transfer efficiency, and a method of estimating the level of exposure to the chemical substance by dermal and/or oral route.

2. Related Background Art

When chemical agents containing chemical substances such as pesticidal compounds are used, the chemical substances tend to be retained as residues in indoor and outdoor objects (such as floors). The level of exposure of a chemical to humans is determined from the residue level, and human safety is evaluated by comparison with the no-observed-adverse-effect level (NOAEL) obtained from results of a mammal toxicity test. For example, the exposure level of a chemical substance through dermal and oral routes is estimated based on the chemical substance residue level in an object, and the estimated value is compared with the NOAEL obtained from the results of a mammal toxicity test, thereby allowing evaluation for human safety (see Japanese Unexamined Patent Publication No.10-182301 (EP0882397A1), for example).

According to the established school of thought, the residual level in objects and dermal transfer efficiency of a chemical substance with repeated use of a chemical agent have been considered as simple uniform functions, with the chemical substance residue level in an object and dermal transferable residue being calculated without considering the temporal change produced by each day of use and each single use, and the exposure level of the chemical substance through dermal and oral routes being estimated based on the calculated value. Thus, when a chemical agent is continuously used several times, the divergence between the estimated and measured values of the chemical substance residue level and dermal transferable residue increases, such that less than satisfactory accuracy is achieved for simulating actual contact migration behavior of the chemical substance.

### SUMMARY OF THE INVENTION

In light of these circumstances, the present inventors discovered that if the chemical substance residue level in an object and dermal transfer efficiency of the chemical substance are considered not as simple uniform functions, but firstly the temporal change is determined not only for the chemical substance residue level in the object but also for the dermal transfer efficiency, and then the chemical substance residue level in the object and the dermal transferable residue are calculated for the time elapsed after use of the chemical substance, divided across each day of use and each single use, integrating the calculated values during the period of time after use of the chemical substance in consideration of the number of days used and number of times used, it is possible to achieve a more accurate simulation of contact migration behavior of the chemical substance, and the present invention was thereupon completed.

Specifically, the present invention provides the following:

1. A method for estimating the dermal transfer efficiency of a chemical substance comprising a step of determining a calculated value for the dermal transfer efficiency for the time elapsed after use of the chemical substance, based on a formula wherein the temporal change in the dermal transfer efficiency is expressed as a function dependent on the −0.5 power of the time, or on a computer program incorporating the formula, and a step of establishing the calculated value determined in the previous step as the estimated dermal transfer efficiency (hereinafter also referred to as “dermal transfer efficiency estimating method of the invention”).

2. The method of 1. above, wherein the formula wherein the temporal change in the dermal transfer efficiency is expressed as a function dependent on the −0.5 power of the time t is the formula: dermal transfer efficiency=a{1+b(t)}^{-0.5} (where a and b represent any constants).

3. A method for estimating dermal transferable residue of a chemical substance comprising a step of determining a calculated value for the dermal transferable residue for the time elapsed after use of the chemical substance, based on a formula wherein the dermal transfer efficiency estimated by the method of 1. above is multiplied by the chemical substance residue level in an object for the time elapsed after use of the chemical substance, or based on a computer program incorporating the formula, and a step of establishing the calculated value determined in the previous step as the estimated dermal transferable residue (hereinafter also referred to as “dermal transferable residue estimating method of the invention”).

4. The method of 3. above, wherein the chemical substance residue level in the object for the time elapsed after use of the chemical substance is the calculated value determined based on a formula wherein the decomposition of the chemical substance is expressed as a first-order formula of time, or based on a computer program incorporating the formula.

5. The method of 4. above, wherein the formula wherein the decomposition of the chemical substance is expressed as a first-order formula of time t is the formula: chemical substance residue level in object=c exp(−dt) (where c and d represent any constants).

6. A method for estimating the dermal transferable residue of a chemical substance comprising a step of determining a calculated value for the dermal transferable residue for the time elapsed after use of the chemical substance, for each single use of the chemical substance, based on a formula wherein the dermal transfer efficiency estimated by the method of 1. above is multiplied by the chemical substance residue level in an object for the time elapsed after use of the chemical substance, or based on a computer program incorporating the formula, and a step of determining the sum of the calculated values determined by the previous step and establishing it as the estimated dermal transferable residue for multiple use of the chemical substance.

7. A method for estimating the dermal transfer residue of a chemical substance, comprising a step of determining a calculated value for the dermal transfer residue of the chemical substance during a period elapsed after used of the chemical substance, based on a formula which integrates the dermal transferable residue estimated by the method of 3. or 6. above for a period of time elapsed after use of the chemical substance, during the period of time, or based on a computer program incorporating the formula, and a step of establishing the calculated value determined in the previous step as the estimated dermal transfer residue of the chemical substance (hereinafter also referred to as “dermal transfer residue estimating method of the invention”).

8. A method for estimating the dermal and/or oral exposure level of a chemical substance, comprising a step of determining a calculated value for the dermal and/or oral exposure level of the chemical substance, during a period of time elapsed after use of the chemical substance, based on a formula which integrates the contact level obtained by multiplying the dermal transferable residue estimated by the method of 3. or 6. above for a period of time elapsed after use of the chemical substance, during the period of time, by a constant which is dependent on the area of contact and route of contact with the chemical substance, or based on a computer program incorporating the formula, and a step of establishing the calculated value determined by the previous step as the estimated dermal and/or oral exposure level of the chemical substance during the period of time elapsed after use of the chemical substance (hereinafter also referred to as “exposure level estimating method of the invention”).

9. A method for estimating the dermal and/or oral exposure level of a chemical substance, wherein the chemical substance is used a plurality of times, comprising a step of determining a calculated value for the dermal and/or oral exposure level of the chemical substance during a period of time elapsed after each single use of the chemical substance, based on a formula which integrates the contact level obtained by multiplying the dermal transferable residue estimated by the method of 3. above for a period of time elapsed after each single use of the chemical substance, during the period of time, by a constant which is dependent on the area of contact and route of contact with the chemical substance, or based on a computer program incorporating the formula, and a step of determining the sum of the calculated values during the period of time elapsed after use of the chemical substance, determined by the previous step, and establishing it as the estimated dermal and/or oral exposure level of the chemical substance during the period of time elapsed after use of the chemical substance.

10. The method according to any one of 1. to 9. above, wherein the chemical substance is a pesticidal compound.

11. A computer program incorporating a formula wherein the temporal change in the dermal transfer efficiency is expressed as a function dependent on the −0.5 power of time t.

12. A computer program incorporating a formula wherein the dermal transfer efficiency estimated by the method of 1. above is multiplied by the chemical substance residue level in an object for a period of time elapsed after use of the chemical substance.

13. A computer program incorporating a formula which integrates the dermal transferable residue estimated by the method of 3. above for a period of time elapsed after use of a chemical substance, during the period of time.

14. A computer program incorporating a formula which integrates the contact level obtained by multiplying the dermal transferable residue estimated by the method of 3. above for a period of time elapsed after use of the chemical substance, during the period of time, by a constant which is dependent on the area of contact and route of contact with the chemical substance.

15. The use of a computer program according to any one of 11. to 14. above for safety evaluation of a chemical substance.

The present invention will become more fully understood from the detailed description given hereinbelow and the accompanying drawings which are given by way of illustration only, and thus are not to be considered as limiting the present invention.

Further scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is a functional block diagram showing the flow of a contact migration behavior predicting method including a dermal transferable residue estimating method, dermal transfer residue estimating method and exposure level estimating method according to an embodiment of the invention, in a ordered relation for a computer program which carries out the method in a computer.

FIG. 2 is a graph showing the behavior of dermal transferable residue for daily use of a household liquid anti-mosquito insecticidal device comprising an insecticidal compound.

FIG. 3 is a set of graphs showing a procedure for establishing the estimated dermal transferable residue for time t for multiple use of a chemical substance, wherein (a) represents the temporal change in the chemical substance residue level and dermal transferable residue of the substance dispersed on the first day, (b) represents the temporal change in the chemical substance residue level and dermal transferable residue of the substance dispersed on the second day, (c) represents the temporal change in the chemical substance residue level and dermal transferable residue of the substance dispersed on the third day, and (d) represents the sum for the three days, i.e., the temporal change in the chemical substance residue level and dermal transferable residue of the total substance dispersed by multiple use.

FIG. 4 is a set of graphs showing the dermal transfer efficiencies in a floor (tatami mat, flooring, carpet) for continuous use of a household liquid anti-mosquito insecticidal device comprising an insecticidal compound, for a period of 12 hours in the first day. The solid circles in the graphs represent the actual measured floor chemical substance residue levels for each elapsed time period (number of elapsed days) ((a) tatami mat, (b) flooring, (c) carpet).

FIG. 5 is a set of graphs showing the results approximating measured values (solid circles) using the formula expressed by the first-order equation listed in Test Example 1, for the dermal transfer efficiencies in a floor ((a) tatami mat, (b) flooring, (c) carpet).

FIG. 6 is a set of graphs showing the results of approximating measured values (solid circles) using the formula wherein the temporal change of the dermal transfer efficiencies in Test Example 1 is represented by a function dependent on the −0.5 power of the time t, for the dermal transfer efficiencies in a floor ((a) tatami mat, (b) flooring, (c) carpet).

FIG. 7 is a graph showing the chemical substance residue levels in a floor (tatami mat) (floor chemical substance residue levels) for continuous use of a household liquid anti-mosquito insecticidal device comprising an insecticidal compound, for a period of 12 hours each day. The solid circles in the graph represent the actual measured floor chemical substance residue levels for each elapsed time period (number of elapsed days). The solid line in the graph represents the results of approximating the measured values (solid circles) by formula (1) and formula (2) in Test Example 1.

FIG. 8 is a graph showing the results of precisely estimating the temporal change in dermal transferable residue by separately determining dermal transferable residue for each electrification and later summing them, based on the present invention.

### DETAILED DESCRIPTION OF THE INVENTION

The present invention is an invention based on the dermal transferable residue estimating method described above, and on the dermal transfer residue estimating method and exposure level estimating method employing it.

According to the invention, “dermal transfer efficiency”, “dermal transferable residue”, “chemical substance residue level in an object” and “contact level” are the values at a specific time (t) during a period of time elapsed after use of the chemical substance, and for example, they are the values at time (t1) between t0 (immediately before use of the chemical agent) and t2 (immediately before the next use of the chemical agent).

Also, “dermal transfer residue” and “exposure level” are the integral values during the period elapsed after use of the chemical substance, and for example, they are the integral values between t0 (immediately before use of the chemical agent) and t2 (immediately before the next use of the chemical agent).

In addition, “dermal transferable residue”, “dermal transfer residue” and “exposure level” are the values determined in the following manner.

(1) Dermal transferable residue=(dermal transfer efficiency)·(chemical substance residue level)

(2) Dermal transfer residue=integral value of dermal transferable residue during period of time elapsed after use of chemical substance (for example, integral value between t0 (immediately before first use of chemical agent) and t2 (immediately before next use of chemical agent))

(3) Exposure level=integral value of contact level during period of time elapsed after use of chemical substance (for example, integral value between t0 (immediately before first use of chemical agent) and t2 (immediately before next use of chemical agent))

In order to solve the problem described above, the dermal transferable residue estimating method of the invention comprises a step of ascertaining the temporal change not only for the chemical substance residue level in an object but also the dermal transfer efficiency, and calculating the dermal transferable residue divided across each day of use and each single use, and a step of establishing the calculated value determined by the previous step as the estimated dermal transfer residue.

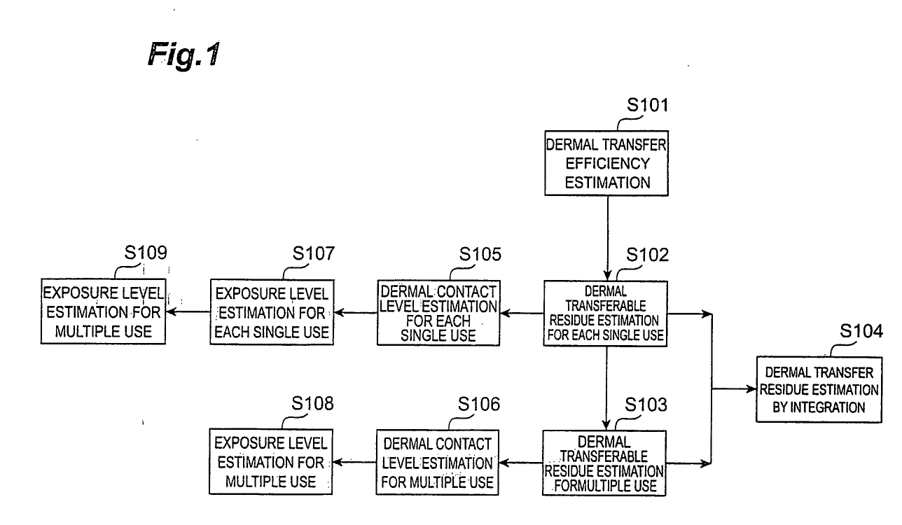

The dermal transferable residue estimating method, dermal transfer residue estimating method and exposure level estimating method will now be explained in detail, with reference to FIG. 1. FIG. 1 is a functional block diagram showing the flow of a contact migration behavior predicting method including a dermal transferable residue estimating method, dermal transfer residue estimating method and exposure level estimating method, in a ordered relation for a computer program which carries out the method in a computer.

When a chemical agent containing a chemical substance such as a pesticidal compound is used, the chemical substance resides in indoor and outdoor objects (for example, floors).

The dermal transfer efficiency is the proportion of a chemical substance adhering to the skin when the skin contacts an object in which the chemical substance is residing, and according to the invention, it is the dermal transfer efficiency for the time elapsed after use of the chemical substance, determined based on a formula wherein the temporal change in the dermal transfer efficiency is expressed as a function dependent on the −0.5 power of the time, or on a computer program incorporating the formula (S101). Here, as the “formula wherein the temporal change in the dermal transfer efficiency is expressed as a function dependent on the −0.5 power of the time” there may be mentioned, specifically, the formula: dermal transfer efficiency=a{1+bt}^{-0.5} (where a and b represent any constants).

Also, since the dermal transferable residue decreases with time after use of the chemical agent, the dermal transferable residue on day d after use is calculated by the following formula (S102).

<Calculation of Dermal Transferable Residue on Day d After Use>

(Dermal transferable residue (mg/m²) on day d after use)=((dermal transfer efficiency (%))/100) (residue level of chemical substance (mg/m²) in object immediately after use)·(1−D)^{d}

Here, D represents the decomposition rate of the residue level of the chemical substance in the object per day, and specifically, for example, the dermal transfer efficiency may be calculated by the following formula: dermal transfer efficiency=a{1+bt}^{-0.5} (where a and b represent any constants).

Thus, the determined calculated value is established as the estimated dermal transfer efficiency (S101), and the calculated value for the dermal transferable residue is determined for the time elapsed after use of a chemical substance for each single use of the chemical substance, based on a formula wherein the dermal transfer efficiency is multiplied by the chemical substance residue level in an object for the time elapsed after a single use of the chemical substance (day d after use, in the example mentioned above), or based on a computer program incorporating the formula. The dermal transferable residue may be estimated by establishing the determined calculated value as the estimated dermal transferable residue (S102).

A method of determining the dermal transferable residue for multiple use of a chemical substance will now be explained.

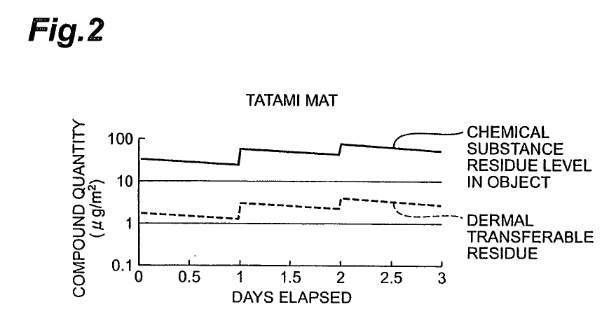

When a chemical agent is used daily, and the change in the residue level of a chemical substance in an object with time is considered to be, for example, uniformly 5%, without considering the change in the dermal transfer efficiency with time, the behavior of the dermal transferable residue (the lower dotted line in FIG. 2) exhibits the same behavior as the residue level of the chemical substance in the object (upper solid line in FIG. 2). That is, simply multiplying the total chemical substance residue level in the object by 5% yields the dermal transferable residue.

Specifically, however, for example, given two days after initial use of the chemical substance, there is a difference between the depth of dispersion of the chemical substance in the object used on the first day, and the depth of dispersion of the chemical substance used on the second day. Consequently, the dermal transfer efficiency of the residue of the chemical substance used on the first day is not exactly equal to the dermal transfer efficiency of the residue of the chemical substance used on the second day. According to the invention, the total of the chemical substance residue level from the initial use of the chemical substance to the second day is not multiplied by the dermal transfer efficiency in a uniform manner, but rather, the residue level of the chemical substance used on the first day and the residue level of the chemical substance used on the second day are separately multiplied by the dermal transfer efficiency in which the temporal change is taken into account.

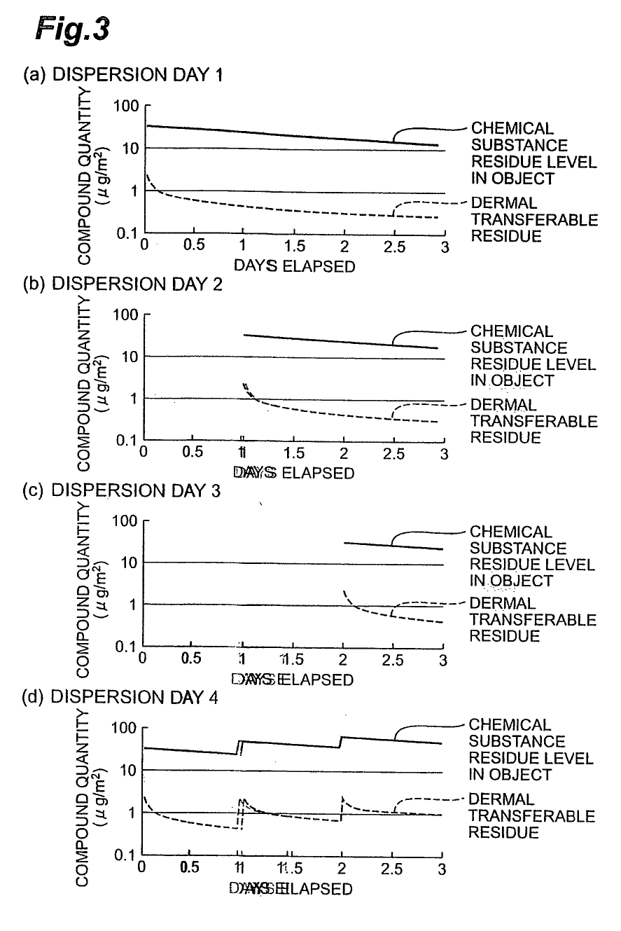

Specifically, in order to consider decomposition of the chemical substance, for example, for the chemical substance used on the first day, the residue level in an object is represented by a formula wherein the decomposition of the chemical substance is expressed as a first-order formula of time t [for example, chemical substance residue level in object=c exp(−dt) (where c and d represent any constants)], and the dermal transfer efficiency is represented by a formula wherein the temporal change thereof is expressed as a function dependent on the −0.5 power of the time t [for example, dermal transfer efficiency=a{1+bt}^{-0.5} (where a and b represent any constants)], after which both are multiplied to determine the dermal transferable residue from initial use of the chemical substance until elapse of time t. The dermal transferable residue of the chemical substance used on the second day and the dermal transferable residue of the chemical substance used on the third day are each determined in the same manner (S102). Next, the sum of the dermal transferable residues at each time, measured as the time elapsed from beginning of the initial use of the chemical substance, is determined and the sum is established as the estimated dermal transferable residue at that time for multiple use of the chemical substance (S103). These procedures are illustrated in FIG. 3.

For example, the dermal transferable residue at a specified time (t1) between t0 (immediately before use of the chemical agent) and t2 (immediately before the next use of the chemical agent), which has been estimated in the manner described above, is integrated across the period of time elapsed after use of the chemical substance (for example, t1=t0˜t2), to determine the calculated value for the dermal transfer residue of the chemical substance during the period of time elapsed after use of the chemical substance (t0˜t2). The determined calculated value is established as the estimated dermal transfer residue of the chemical substance, to estimate the dermal transfer residue of the chemical substance (S104).

A method for determining the dermal and/or oral exposure level of a chemical substance, for multiple use of the chemical substance, will now be explained.

A human is exposed by dermal and/or oral routes to a chemical substance in an amount corresponding to the residue level of the chemical substance in an object. When this phenomenon is represented by a numerical formula, for example, the dermal contact level of the chemical substance based on the chemical substance residue level in the object (hereinafter also referred to as dermal contact level) and the contact level through the mouth (hereinafter also referred to as oral contact level) may be obtained by multiplying the dermal transferable residue estimated in the manner described above by a constant which is dependent on the area of contact and route of contact with the chemical substance, and specifically, they may be calculated by the following formulas, for example (S105, S106).

<Calculation of Dermal Contact Level>

(Dermal contact level (mg/kg/day))=(chemical substance residue level in object (mg/m²))·((dermal transfer efficiency (%))/100)·(dermal contact area per day (m²/day))÷(weight [kg])

<Calculation of Oral Contact Level>

(Oral contact level (mg/kg/day))=(chemical substance residue level in object (mg/m²))·((dermal transfer efficiency (%))/100)·(hand contact area per day (m²/day))·((oral migration rate (%))/100)÷(weight [kg])+(food or utensil residue level (mg/m²))·(food intake level or utensil use level per day (m²/day))·((oral migration rate (%))/100)÷(weight (kg))

Here, the chemical substance residue level in the object and the food or utensil residue level may be determined either by projection or by using the chemical agent and measuring the chemical substance residue level. The chemical substance residue level may also be determined from a formula wherein decomposition of the chemical substance is expressed as a first-order formula of time t, as above.

If the dermal transferable residue for each single use (S102) is used in the above calculations as the dermal transferable residue, the contact level for each single use can be obtained (S105). On the other hand, if the dermal transferable residue for multiple use (S103) is used in the above calculations as the dermal transferable residue, the contact level for multiple use can be obtained (S106).

The calculated value for the dermal and/or oral exposure level of the chemical substance, for multiple use of the chemical substance, is obtained, for example, using the dermal contact level or oral contact level, as follows.

The dermal contact level and/or oral contact level during a divided period of time including, for example, a specified time (t1) between t0 (immediately before first use of the chemical agent) and t2 (immediately before the second use of the chemical agent), which was obtained in the manner described above (one day, in the aforementioned example), is integrated across the period of time elapsed after first use of the chemical substance (for example, t1=t0˜t2), to determine the calculated value for the dermal and/or oral exposure level of the chemical substance during the period of time elapsed after use of the chemical substance (t0˜t2), (S107).

<Calculation of Dermal Exposure Level>

$$\begin{matrix}\text{dermal exposure level (mg/kg)} = \frac{1}{{t}_{0} - {t}_{2}} {\int }_{t0}^{t2} ( \text{chemical} \\ \text{substance residue in object} \\ \text{by first use} ( mg \text{/} {m}^{2} ) ) \times \\ ( \text{dermal transfer efficiency} \\ \text{for first use(\%)} ) / 100 ⅆ t \times \\ ( \text{dermal contact area from} \\ {t}_{0} to {t}_{2} ( {m}^{2} ) ) \div ( weight ( kg ) )\end{matrix}$$

<Calculation of Oral Exposure Level>

$$\begin{matrix}\text{oral exposure level (mg/kg)} = \frac{1}{{t}_{0} - {t}_{2}} {\int }_{t0}^{t2} \text{(chemical substance} \\ \text{residue in object by first use} \\ ( mg \text{/} {m}^{2} ) ) \times \text{(dermal transfer} \\ efficiency for first use ( \% ) ) / 100 \\ ⅆ t \times ( hand contact area from \\ {t}_{0} to {t}_{2} ( {m}^{2} ) ) \times ( oral maigration \\ rate ( \% ) / 100 ) \div ( weight ( kg ) )\end{matrix}$$

Next, the calculated value for the dermal and/or oral exposure level of the chemical substance during the period of time including a specified time (t3) between t2 (immediately before second use of the chemical agent) and t4 (immediately before the third use of the chemical agent) is obtained as follows (S107).

<Calculation of Dermal Exposure Level>

$$\begin{matrix}dermal exposure level ( mg \text{/} kg ) = \{ \frac{1}{{t}_{2} - {t}_{4}} {\int }_{t2}^{t4} ( chemical \\ substance residue in object by \\ second use ( mg \text{/} {m}^{2} ) ) \times \\ ( dermal transfer efficiency for \\ second use ( \% ) ) / 100 ⅆ t + \\ \frac{1}{{t}_{2} - {t}_{4}} {\int }_{t2}^{t4} ( chemical substance \\ residue in object by first use \\ ( mg \text{/} {m}^{2} ) ) \times ( dermal transfer \\ efficiency for first use ( \% ) ) / \\ 100 ⅆ t \} \times ( dermal contact area \\ from {t}_{2} to {t}_{4} ( {m}^{2} ) ) \div \\ ( weight ( kg ) )\end{matrix}$$

<Calculation of Oral Exposure Level>

$$\begin{matrix}oral exposure level ( mg \text{/} kg ) = \{ \frac{1}{{t}_{2} - {t}_{4}} {\int }_{t2}^{t4} ( chemical substance \\ residue in object by second use \\ ( mg \text{/} {m}^{2} ) ) \times ( dermal transfer \\ efficiency for second use ( \% ) ) / \\ 100 ⅆ t + \frac{1}{{t}_{2} - {t}_{4}} {\int }_{t2}^{t4} ( chemical \\ substance residue in object by first \\ use ( mg \text{/} {m}^{2} ) ) \times ( dermal transfer \\ efficiency for first use ( \% ) ) / \\ 100 ⅆ t \} \times ( hand contact area from \\ {t}_{2} to {t}_{4} ( {m}^{2} ) ) \times ( oral maigration \\ rate ( \% ) / 100 ) \div ( weight ( kg ) ) + \\ \{ \frac{1}{{t}_{2} - {t}_{4}} {\int }_{t2}^{t4} ( food or utensil residue \\ level for second use ( mg \text{/} {m}^{2} ) ⅆ t + \\ \frac{1}{{t}_{2} - {t}_{4}} {\int }_{t2}^{t4} ( food or utensil residue \\ level for first use ( mg \text{/} {m}^{2} ) ⅆ t \} \times \\ ( food intake level or utensil use \\ level from {t}_{2} to {t}_{4} ) \times ( oral \\ migration rate ( \% ) / 100 ) \div \\ ( weight ( kg ) )\end{matrix}$$

Next, the calculated value for the dermal and/or oral exposure level of the chemical substance during the period of time including a specified time (t5) between t4 (immediately before third use of the chemical agent) and t6 (immediately before the fourth use of the chemical agent) is obtained as follows (S107).

<Calculation of Dermal Exposure Level>

$$\begin{matrix}dermal exposure level ( mg \text{/} kg ) = \{ \frac{1}{{t}_{4} - {t}_{6}} {\int }_{t4}^{t6} ( chemical substance \\ residue in object by third use \\ ( mg \text{/} {m}^{2} ) ) \times ( dermal transfer \\ efficiency for third use ( \% ) ) / \\ 100 ⅆ t + \frac{1}{{t}_{4} - {t}_{6}} {\int }_{t4}^{t6} ( chemical \\ substance residue in object by \\ second use ( mg \text{/} {m}^{2} ) ) \times ( dermal \\ transfer efficiency for second \\ use ( \% ) ) / 100 ⅆ t + \frac{1}{{t}_{4} - {t}_{6}} \\ {\int }_{t4}^{t6} ( chemical substance residue \\ in object by first use \\ ( mg \text{/} {m}^{2} ) ) \times ( dermal transfer \\ efficiency for first use ( \% ) ) / \\ 100 ⅆ t \} \times ( dermal contact area \\ from {t}_{4} to {t}_{6} ( {m}^{2} ) ) \div \\ ( weight ( kg ) )\end{matrix}$$

<Calculation of Oral Exposure Level>

$$\begin{matrix}oral exposure level ( mg \text{/} kg ) = ( \frac{1}{{t}_{4} - {t}_{6}} {\int }_{4}^{6} ( chemical substance \\ residue in object by third use \\ ( mg \text{/} {m}^{2} ) ) \times ( dermal transfer \\ efficiency for third use ( \% ) ) / \\ 100 ⅆ t + \frac{1}{{t}_{4} - {t}_{6}} {\int }_{t4}^{t6} ( chemical \\ substance residue in object by \\ second use ( mg \text{/} {m}^{2} ) ) \times ( dermal \\ transfer efficiency for second use \\ ( \% ) ) / 100 ⅆ t + \frac{1}{{t}_{4} - {t}_{6}} {\int }_{t4}^{t6} ( chemical \\ substance residue in object by \\ first use ( mg \text{/} {m}^{2} ) ) \times ( dermal \\ transfer efficiency for first use \\ ( \% ) ) / 100 ⅆ t \} \times ( hand contact area \\ from {t}_{4} to {t}_{6} ( {m}^{2} ) ) \times ( oral \\ maigration rate ( \% ) / 100 ) \div \\ ( weight ( kg ) ) + \{ \frac{1}{{t}_{4} - {t}_{6}} {\int }_{t4}^{t6} ( food \\ or utensil residue level for third \\ use ( mg \text{/} {m}^{2} ) ⅆ t + \frac{1}{{t}_{4} - {t}_{6}} {\int }_{t4}^{t6} ( food \\ or utensil residue level for second \\ use ( mg \text{/} {m}^{2} ) ⅆ t + \frac{1}{{t}_{4} - {t}_{6}} {\int }_{t4}^{t6} ( food \\ or utensil residue level for first \\ use ( mg \text{/} {m}^{2} ) ⅆ t \} \times ( food intake \\ level or utensil use level from {t}_{4} \\ to {t}_{6} ) \times ( oral migration rate ( \% ) / \\ 100 ) \div ( weight ( kg ) )\end{matrix}$$

The calculated value for the dermal and/or oral exposure level of the chemical substance during the next periods of time can be obtained in a same manner.

Finally, summation of the calculated values for the dermal exposure level and/or summation of the calculated values for the oral exposure level are calculated respectively.

By establishing the determined calculated level (each summation) as the estimated dermal and/or oral exposure level for multiple use of the chemical substance, it is possible to estimate the dermal and/or oral exposure level of the chemical substance during the period of time elapsed after first use of the chemical substance (S109).

The estimated dermal and/or oral exposure level for multiple use of the chemical substance also can be obtained similarly integrating not the contact level for each single use obtained in Step S105 but the contact level for multiple use obtained in Step S106 (S108).

By reflecting the temporal change in the dermal transferable residue for multiple use of a chemical substance according to the invention as described above, it is possible to more accurately represent the behavior of the dermal transferable residue, to allow simulations with different chemical substance contact migration behaviors to be carried out regardless of the material in question. Thus, a calculated value is determined for the dermal transfer residue of the chemical substance or the dermal and/or oral exposure level of the chemical substance, during a period of time elapsed after use of the chemical substance, and this is established as the estimated dermal transfer residue or exposure level for multiple use of the chemical substance. Also, the established exposure level is compared with the no-observed effect level obtained from results of a mammal toxicity test, to evaluate safety in humans.

### EXAMPLES

The present invention will now be explained in more detail through the following test examples, although these test examples are in no way limitative on the invention.

### Test Example 1 (Dermal Transfer Efficiency)

The temporal relationship of the dermal transfer efficiency of the invention will now be explained.

When a pesticidal compound (a household insecticidal agent in this test) is dispersed indoors, the dermal transfer efficiency is highest immediately after dispersion, and decreases with the passage of time.

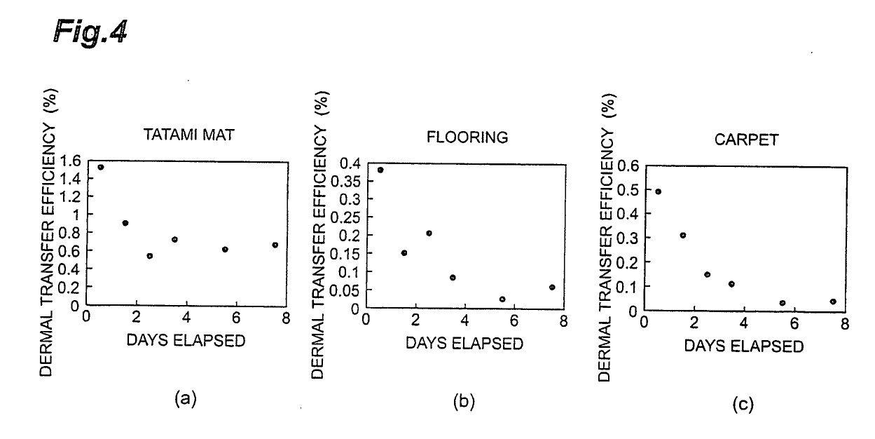

The experimental method used for measurement of the dermal transfer efficiency was the method described in Japanese Unexamined Patent Publication No. 10-182301 (EP0882397A1). Specifically, a weight is placed on a denim cloth at the same pressure as the pressure of contact of a human with the object, and the denim cloth is pulled over the floor at a speed similar to human motion. The denim and floor are analyzed and the chemical substance levels in the denim and floor are calculated, with the dermal transfer efficiency being determined from the ratio. FIG. 4 shows the actual results for the dermal transfer efficiency obtained in this manner.

As shown in FIG. 4, it can be judged that the dermal transfer efficiency is related to time, but the specific relationship cannot be easily ascertained and is unclear. For example, in a formula wherein the temporal change in the dermal transfer efficiency is expressed as a first-order function of time t (for example, dermal transfer efficiency=a+bt), there is obviously a large discrepancy between the calculated and measured values for the dermal transfer efficiency, and therefore it cannot be expressed by this formula.

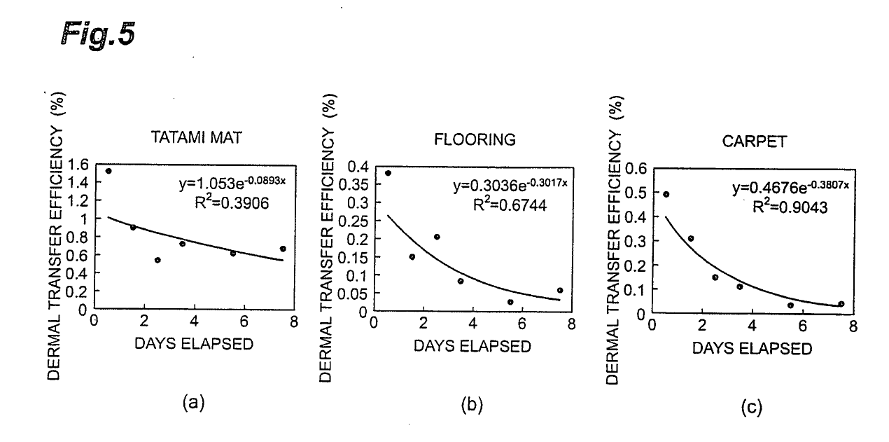

FIG. 5 shows the results of representing the temporal change in dermal transfer efficiency by a first-order formula (for example, dermal transfer efficiency=a(exp(−bt)), which is often used to represent the behavior in the environment.

As clearly seen in FIG. 5, the correlation coefficient (r²) is 0.3906 when the object is a tatami mat, 0.6744 when the object is a flooring and 0.9043 when the object is a carpet, indicating a particularly low correlation for a tatami mat and flooring. Thus, it was not possible to adequately express the change in the dermal transfer efficiency with time for the three typical floor materials.

Next, it was decided to represent the temporal change in the dermal transfer efficiency by a formula wherein the change is expressed as a function which is dependent on the −0.5 power of time t. In the case of the dermal transfer efficiency measurement method described above, for example, the floor is wiped with a denim cloth and the dermal transfer efficiency is calculated from the ratio between the chemical substance residue level in the floor and the chemical substance level adhering to the denim cloth. The residue behavior of the chemical substance in the floor may be considered as follows. When the chemical substance is deposited on the floor, the chemical substance begins to diffuse into the floor as time passes. The depth (e, units: m) of diffusion may be represented by the following formula:
e=2\sqrt{D_{c}t}
(where D_{c} represents the diffusion coefficient (m²/day) and t represents the elapsed time (days).

On the other hand, the chemical substance level adhering to the denim cloth corresponds to the chemical substance level adhering to skin, etc. when a constant pressure is applied to the surface of the floor causing adhesion to the skin, etc, and the depth contributing to migration of the chemical substance from the floor surface to the skin, etc. (x, units=m) does not vary with time so long as the pressure is constant. Consequently, when a pressure is applied and skin, etc. is contacted with the floor surface, only the chemical substance at distance x, from among the chemical substance diffused to distance e from the floor surface, contributes to the dermal transfer efficiency. This may be expressed by a formula, as follows:

Proportion of depth of chemical substance contributing to dermal transfer efficiency=x/2\sqrt{D_{c}t}

When considering the mechanism of diffusion of the chemical substance into the interior of the floor, the change in the dermal transfer efficiency with time can be represented by the following formula:

Dermal transfer efficiency=a/\sqrt{1+b×time}

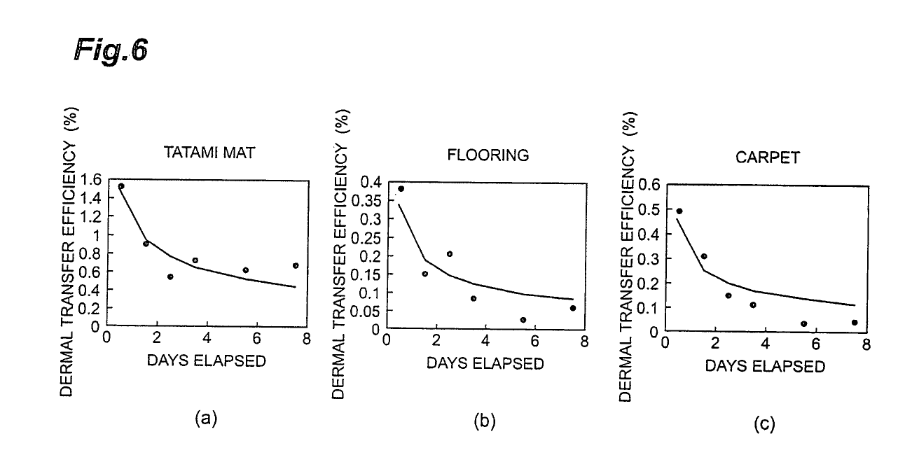

FIG. 6 shows the results for the temporal change in dermal transfer efficiency represented by a formula wherein the change is expressed as a function dependent on the −0.5 power of time t. The constant a, b, the correlation coefficients (r²) and r are shown in Table 1.

As is clear from FIG. 6 and Table 1, the correlation coefficient (r²) is 0.847 when the object is a tatami mat, 0.887 when the object is a flooring and 0.965 when the object is a carpet, indicating a high correlation for the tatami mat, flooring and carpet. Thus, the temporal change in the dermal transfer efficiency could be adequately expressed for the three typical floor materials.

TABLE 1

| Tatami mat | Flooring | Carpet |
|---|---|---|
| a = 2.68 | a = 5.01 | a = 9.73 |
| b = 4.56 | b = 457 | b = 1020 |
| r = 0.920 | r = 0.942 | r = 0.982 |
| r² = 0.847 | r² = 0.887 | r² = 0.965 |

It is therefore possible to represent the behavior of the dermal transfer efficiency for a period of time elapsed after use of a chemical substance, based on a formula wherein the temporal change in the dermal transfer efficiency is expressed as a function dependent on the −0.5 power of the time, to allow simulations with different chemical substance contact migration behaviors to be carried out regardless of the material in question.

### Test Example 2 (Dermal Transferable Residue)

The temporal relationship of the dermal transferable residue of the invention will now be explained through a concrete calculation method.

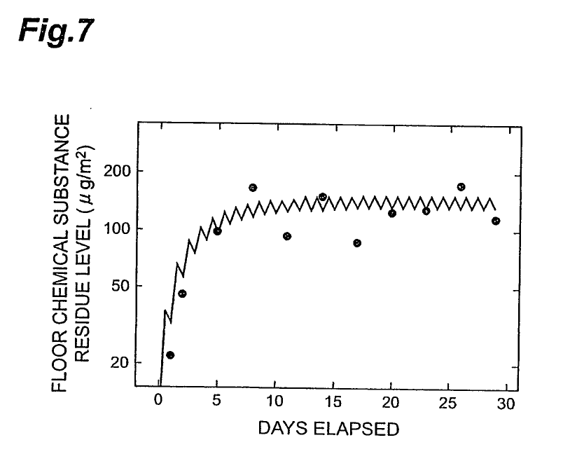

The solid circles in FIG. 7 represent chemical substance residue levels in the floor (hereinafter referred to as floor chemical substance residue levels) when a household liquid anti-mosquito insecticidal device comprising an insecticidal compound is used continuously for 12 hours each day. The solid circles are the floor chemical substance residue values actually measured for a tatami mat as the floor material. Since the liquid anti-mosquito insecticidal device is electrified from time 0 to day 0.5 (first electrification), the floor chemical substance residue level increases, and then decreases due to decomposition on the floor from day 0.5 to day 1. On the following day as well, the liquid anti-mosquito insecticidal device is electrified from day 1 to day 1.5 (second electrification), and therefore the floor chemical substance residue level increases and then decreases from day 1.5 to day 2. This cycle is repeated continuously. If the rate of increase of the liquid anti-mosquito insecticidal device-released insecticidal compound in the floor is represented as E (micro g/m²/day) and the decomposition rate of the insecticidal compound on the floor is represented as k(/day), the floor chemical substance residue level [(R(t), micro g/m²] may be expressed by the following formula (1):

Formula (1)

The floor chemical substance residue level for time t from day (n−1) to day (n−0.5), for the nth electrification, is expressed by the formula:

$$\begin{matrix}R ( t ) = ( E / k ) ( 1 - {ⅇ}^{- k / 2} ) \frac{( 1 - {ⅇ}^{- k ( n - 1 )} )}{( 1 - {ⅇ}^{- k} )} {ⅇ}^{- k ( t - n + 1.5 )} + \\ ( E / k ) ( 1 - {ⅇ}^{- k ( t - n + 1 )} )\end{matrix}$$

The floor chemical substance residue level for time t from day (n−0.5) to day (n), for the nth electrification, is expressed by the formula:

$$R ( t ) = ( E / k ) ( 1 - {ⅇ}^{- k / 2} ) \frac{( 1 - {ⅇ}^{- kn} )}{( 1 - {ⅇ}^{- k} )} {ⅇ}^{- k ( t - n + 0.5 )}$$

Upon approximating these formulas to the solid circles in FIG. 7, E and k were 81.8 micro g/m²/day and 0.295/day, respectively.

On the other hand, the dermal transfer efficiency for a tatami mat [TE(t), %] may be represented by the following formula (2), based on the results of Test Example 1. The dermal transfer efficiency after electrification for 0.5 hour was 1.5%.

Formula (2)
TE(t) for tatami=2.68/\sqrt{1+4.56t}

The dermal transferable residue is the product of the floor chemical substance residue level and the dermal transfer efficiency.

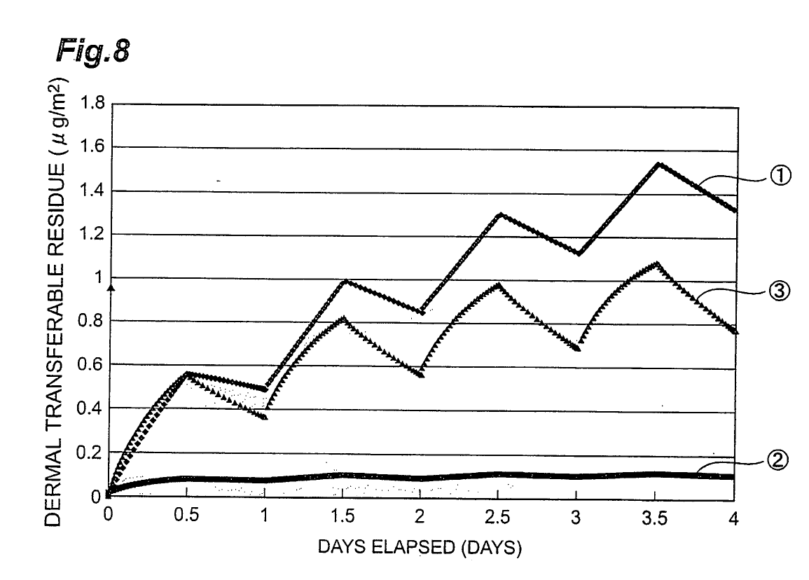

If the dermal transferable residue is assumed to be constant (for example, 1.5%) regardless of time, the floor chemical substance residue level derived from formula (1) may be multiplied by 1.5% to calculated the dermal transferable residue. This will give the plot of the solid diamonds (♦) in FIG. 8 ([1]). In actuality, as is clear from the results of Test Example 1, the dermal transfer efficiency decreases with time, based on the function which is dependent on the −0.5 power of time. That is, the plot of the solid diamonds in FIG. 8 ([1]) cannot be considered a more precise dermal transferable residue. Also, if the dermal transferable residue derived from formula (2) is simply multiplied by the floor chemical substance residue level derived from formula (1), the dermal transferable residue becomes the plot of solid squares (▪) in FIG. 8 ([2]). In actuality, although the dermal transfer efficiency of the insecticidal compound released from the liquid anti-mosquito insecticidal device electrified the second time should be 1.5% after 0.5 day from release (1.5 day from the first electrification), when t=1.5 is used for formula (2), the dermal transfer efficiency becomes 0.957%, and therefore the estimated dermal transferable residue is clearly too small.

Therefore, a procedure is carried out wherein the dermal transferable residue of the compound dispersed on the first day and the dermal transferable residue of the compound dispersed on the second day are distinguished and later summed (see FIG. 3), to determine the dermal transferable residue which is shown as the plot of solid triangles (▴) in FIG. 8 ([3]). This may be represented by the following formulas.

The dermal transferable residue [TR(t), micro g/m²] for time t from day (n−1) to day (n−0.5), for the nth electrification, is expressed by the formula:

$$\begin{matrix}TR ( t ) = E / k ( 1 - {ⅇ}^{- k / 2} ) {ⅇ}^{- k ( t - 0.5 )} \times TE ( t ) / 100 + & ⋯ & 1 st evaporation \\ E / k ( 1 - {ⅇ}^{- k / 2} ) {ⅇ}^{- k ( t - 1.5 )} \times TE ( t - 1 ) / 100 + & ⋯ & 2 nd evaporation \\ ⋯ & & \\ E / k ( 1 - {ⅇ}^{- k / 2} ) {ⅇ}^{- k ( t - n + 1.5 )} \times TE ( t - n + 2 ) / 100 + & ⋯ & ( n - 1 ) th evaporation \\ E / k ( 1 - {ⅇ}^{- k ( t - n + 1 )} ) \times TE ( t - n + 1 ) / 100 & ⋯ & nth evaporation\end{matrix}$$

The dermal transferable residue for time t from day (n−0.5) to day (n), for the nth electrification, is expressed by the formula:

$$\begin{matrix}TR ( t ) = E / k ( 1 - {ⅇ}^{- k / 2} ) {ⅇ}^{- k ( t - 0.5 )} \times TE ( t ) / 100 + & ⋯ & 1 st evaporation \\ E / k ( 1 - {ⅇ}^{- k / 2} ) {ⅇ}^{- k ( t - 1.5 )} \times TE ( t - 1 ) / 100 + & ⋯ & 2 nd evaporation \\ ⋯ & & \\ E / k ( 1 - {ⅇ}^{- k / 2} ) {ⅇ}^{- k ( t - n + 1.5 )} \times TE ( t - n + 2 ) / 100 + & ⋯ & ( n - 1 ) th evaporation \\ E / k ( 1 - {ⅇ}^{- k / 2} ) {ⅇ}^{- k ( t - n + 0.5 )} \times TE ( t - n + 1 ) / 100 & ⋯ & nth evaporation\end{matrix}$$

In other words, on day 2 from the first electrification, for example, the depth of diffusion into the tatami mat of the insecticidal compound used on the first day differs from the depth of diffusion of the insecticidal compound used on the second day, and therefore the dermal adhesion of the floor residue of the insecticidal compound diffused on day 1 is not exactly the same as the dermal adhesion of the floor residue of the insecticidal compound diffused on day 2. Thus, by distinguishing the dermal transferable residue according to each electrification and later taking the sum, it is possible to estimate the temporal change in the dermal transferable residue in a more precise manner.

According the invention, the aforementioned construction allows the chemical substance residue level in an object and the dermal transferable residue, resulting from continued multiple repeated use of a chemical agent, to be approximated for practical situations, thereby making possible a more precise simulation of the contact migration behavior of the chemical substance. In addition, it allows the safety of chemical substances such as pesticidal compounds for the human body, especially by dermal and oral exposure, to be evaluated with high precision, in order to permit the aforementioned simulation to be easily repeated under different conditions for formulation of the chemical substance, and thus facilitate formulation of the chemical agent in a highly safe manner in accordance with the intended purpose.

From the invention thus described, it will be obvious that the invention may be varied in many ways. Such variations are not to be regarded as a departure from the spirit and scope of the invention, and all such modifications as would be obvious to one skilled in the art are intended for inclusion within the scope of the following claims.

1. A method for estimating the dermal transfer efficiency of a chemical substance comprising a step of determining a calculated value for the dermal transfer efficiency for the time elapsed after use of the chemical substance, based on a formula wherein the temporal change in the dermal transfer efficiency is expressed as a function dependent on the −0.5 power of the time, or on a computer program incorporating the formula, and a step of establishing the calculated value determined in the previous step as the estimated dermal transfer efficiency.

2. The method of claim 1, wherein the formula wherein the temporal change in the dermal transfer efficiency is expressed as a function dependent on the −0.5 power of the time t is the formula: dermal transfer efficiency=a{1+b(t)}^{-0.5}.

3. A method for estimating dermal transferable residue of a chemical substance comprising a step of determining a calculated value for the dermal transferable residue for the time elapsed after use of the chemical substance, based on a formula wherein the dermal transfer efficiency estimated by the method of claim 1 is multiplied by the chemical substance residue level in an object for the time elapsed after use of the chemical substance, or based on a computer program incorporating the formula, and a step of establishing the calculated value determined in the previous step as the estimated dermal transferable residue.

4. The method of claim 3, wherein the chemical substance residue level in the object for the time elapsed after use of the chemical substance is the calculated value determined based on a formula wherein the decomposition of the chemical substance is expressed as a first-order formula of time, or based on a computer program incorporating the formula.

5. The method of claim 4, wherein the formula wherein the decomposition of the chemical substance is expressed as a first-order formula of time t is the formula: chemical substance residue level in object=c exp(−d(t)) (where c and d represent any constants);

6. A method for estimating dermal transferable residue of a chemical substance comprising a step of determining a calculated value for the dermal transferable residue for the time elapsed after use of the chemical substance, for each single use of the chemical substance, based on a formula wherein the dermal transfer efficiency estimated by the method of claim 1 is multiplied by the chemical substance residue level in an object for the time elapsed after use of the chemical substance, or based on a computer program incorporating the formula, and a step of determining the sum of the calculated values determined by the previous step and establishing it as the estimated dermal transferable residue for multiple use of the chemical substance.

7. A method for estimating the dermal transfer residue of a chemical substance, comprising a step of determining a calculated value for the dermal transfer residue of the chemical substance during a period elapsed after used of the chemical substance, based on a formula which integrates the dermal transferable residue estimated by the method of claim 3 for a period of time elapsed after use of the chemical substance, during the period of time, or based on a computer program incorporating the formula, and a step of establishing the calculated value determined in the previous step as the estimated dermal transfer residue of the chemical substance.

8. A method for estimating the dermal transfer residue of a chemical substance, comprising a step of determining a calculated value for the dermal transfer residue of the chemical substance during a period elapsed after used of the chemical substance, based on a formula which integrates the dermal transferable residue estimated by the method of claim 6 for a period of time elapsed after use of the chemical substance, during the period of time, or based on a computer program incorporating the formula, and a step of establishing the calculated value determined in the previous step as the estimated dermal transfer residue of the chemical substance.

9. A method for estimating the dermal and/or oral exposure level of a chemical substance, comprising a step of determining a calculated value for the dermal and/or oral exposure level of the chemical substance, during a period of time elapsed after use of the chemical substance, based on a formula which integrates the contact level obtained by multiplying the dermal transferable residue estimated by the method of claim 3 for a period of time elapsed after use of the chemical substance, during the period of time, by a constant which is dependent on the area of contact and route of contact with the chemical substance, or based on a computer program incorporating the formula, and a step of establishing the calculated value determined by the previous step as the estimated dermal and/or oral exposure level of the chemical substance during the period of time elapsed after use of the chemical substance.

10. A method for estimating the dermal and/or oral exposure level of a chemical substance, comprising a step of determining a calculated value for the dermal and/or oral exposure level of the chemical substance, during a period of time elapsed after use of the chemical substance, based on a formula which integrates the contact level obtained by multiplying the dermal transferable residue estimated by the method of claim 6 for a period of time elapsed after use of the chemical substance, during the period of time, by a constant which is dependent on the area of contact and route of contact with the chemical substance, or based on a computer program incorporating the formula, and a step of establishing the calculated value determined by the previous step as the estimated dermal and/or oral exposure level of the chemical substance during the period of time elapsed after use of the chemical substance.

11. A method for estimating the dermal and/or oral exposure level of a chemical substance, wherein the chemical substance is used a plurality of times, comprising a step of determining a calculated value for the dermal and/or oral exposure level of the chemical substance during a period of time elapsed after each single use of the chemical substance, based on a formula which integrates the contact level obtained by multiplying the dermal transferable residue estimated by the method of claim 3 for a period of time elapsed after each single use of the chemical substance, during the period of time, by a constant which is dependent on the area of contact and route of contact with the chemical substance, or based on a computer program incorporating the formula, and a step of determining the sum of the calculated values during the period of time elapsed after use of the chemical substance, determined by the previous step, and establishing it as the estimated dermal and/or oral exposure level of the chemical substance during the period of time elapsed after use of the chemical substance.

12. The method according to claim 1, wherein the chemical substance is a pesticidal compound.

13. A computer program incorporating a formula wherein the temporal change in the dermal transfer efficiency is expressed as a function dependent on the −0.5 power of time t.

14. A computer program incorporating a formula wherein the dermal transfer efficiency estimated by the method of claim 1 is multiplied by the chemical substance residue level in an object for a period of time elapsed after use of the chemical substance.

15. A computer program incorporating a formula which integrates the dermal transferable residue estimated by the method of claim 3 for a period of time elapsed after use of a chemical substance, during the period of time.

16. A computer program incorporating a formula which integrates the contact level obtained by multiplying the dermal transferable residue estimated by the method of claim 3 for a period of time elapsed after use of the chemical substance, during the period of time, by a constant which is dependent on the area of contact and route of contact with the chemical substance.

17. The use of a computer program according to claim 13 for safety evaluation of a chemical substance.
